(19) United States
(12) Patent Application Publication
(10) Pub. No.: US 2022/0395514 A1
(43) Pub. Date: Dec. 15, 2022

(54) LOW-DOSE CELECOXIB PREPARATION

(71) Applicant: ARISSA PHARMA LTD, Shanghai (CN)

(72) Inventors: Hong Sun, Shanghai (CN); Tao Wang, Shanghai (CN); Yan Hu, Shanghai (CN); Fenrong Luo, Shanghai (CN); Jinxiang Zhan, Shanghai (CN); Yungen Xie, Shanghai (CN); Jiawei Qi, Shanghai (CN); Luwei Zhao, Shanghai (CN)

(21) Appl. No.: 17/622,669

(22) PCT Filed: Jun. 26, 2019
(86) PCT No.: PCT/CN2019/093044

§ 371 Date: Jun. 29, 2022

(51) Int. Cl. A61K 31/635; A61K 9/00; A61K 9/48

(52) U.S. Cl. CPC A61K 31/635; A61K 9/0053; A61K 9/4833; A61K 9/4858; A61K 9/4866; A61K 9/485

(57) ABSTRACT

The disclosed invention relates to a low-dose celecoxib oral formulation, and a preparation method therefor, which is characterized in that the strength of the formulation is 60-90% of the original strength of the commercial celecoxib product, and the celecoxib formulation with such reduced strength is bioequivalent to the commercial celecoxib product. The celecoxib formulation can be used for the treatment of mild to moderate pain and mild to moderate chronic pain.

### FIELD OF DISCLOSURE

The disclosure relates to the field of formulation development; in particular, it relates to the preparation and application of a low strength (or: low-dose) celecoxib oral formulation that can be used to treat pain and inflammation, which includes nanoparticle preparation, formulation stabilization and manufacturing process.

### BACKGROUND OF DISCLOSURE

Celecoxib belongs to a class of non-steroidal anti-inflammatory drugs (“NSAIDs”), and is mainly used to treat osteoarthritis, rheumatoid arthritis and some acute pains. The commercial product Celebrex® was developed by Pfizer and marketed in the United State and many other countries around the world. Oral formulations are in the dosage of capsules, with the following strengths: 50 mg, 100 mg, 200 mg, 400 mg.

Despite excellent efficacy, celecoxib has risks of cardiovascular events and gastrointestinal irritation. Commercial products carry the U.S. Food and Drug Administration (“FDA”) safety black-box warning, indicating that when the treatment goals are met and “where possible, reduce the dosage and duration of use”.

Therefore, celecoxib has a large space for formulation optimization with respect to safety. There is an urgent need in the art to provide a new celecoxib formulation that is bioequivalent to the commercial celecoxib product but with reduced amount or reduced strength.

### SUMMARY OF DISCLOSURE

The purpose of the present disclosure is to provide a new celecoxib formulation that can reduce the drug amount in the product while achieving bioequivalence as compared to the commercial celecoxib product.

In the first aspect of the disclosure, there is provided an oral celecoxib formulation, the strength of which is at 60-90% of the original strength of the commercial celecoxib product while keeping bioequivalent to the commercial product.

In another preferred embodiment, the formulation dosage is in the form of tablets, capsules, granules, and suspensions.

In another preferred embodiment, the particle size of celecoxib in the formulation is maintained at not greater than 160 nm for D50, and not greater than 300 nm for D90. In another preferred embodiment, the particle size of celecoxib in the formulation intermediate or the nanoparticulate dispersion, is maintained at not greater than 160 nm for D50, and not greater than 300 nm for D90, both of which are for at least 5 days, more preferably 7 days, and even more preferably 7-15 days.

In another preferred embodiment, when the dosage is in the form of tablets, capsules or granules, based on total weight calculation, the formulation comprises sodium dodecyl sulfate at 0.5-12% w/w, more preferably 2-10% w/w, and even more preferably 4-8% w/w.

In another preferred embodiment, when the dosage is in the form of tablets, capsules or granules, based on weight calculation, the formulation comprises polyvinylpyrrolidone at 0.5-7% w/w, more preferably 0.5-5% w/w, and even more preferably 0.5-3% w/w.

In another preferred embodiment, when the dosage is in the form of tablets, capsules or granules, based on weight calculation, the formulation comprises hydroxypropyl cellulose (HPC) at 0.5-7% w/w, more preferably 0.5-5% w/w, and even more preferably 0.5-3% w/w.

In another preferred embodiment, when the dosage is in the form of tablets, capsules or granules, based on weight calculation, the formulation comprises hydroxypropyl methyl cellulose (HPMC) at 0.5-7% w/w, more preferably 0.5-5% w/w, and even more preferably 0.5-3% w/w.

In another preferred embodiment, when the dosage is in the form of tablets, capsules or granules, based on weight calculation, the formulation comprises sucrose at 10-70% w/w, more preferably 10-50% w/w, and even more preferably 10-30% w/w.

In another preferred embodiment, wherein the dosage is in the form of tablets, capsules or granules, the strength include: 40 mg, 80 mg, 160 mg, 320 mg, which corresponds respectively to the strengths of the commercial celecoxib product: 50 mg, 100 mg, 200 mg, 400 mg.

In another preferred embodiment, wherein dosage is in the form of tablets, capsules or granules, the formulation further comprises one or more of pharmaceutically acceptable excipients: fillers, disintegrants, binders, glidants, lubricants.

In another preferred embodiment, wherein the dosage is in the form of tablets, capsules or granules, and the strength is in the range of 40-80 mg, the dissolution of celecoxib is not less than 30% in 30 minutes and not less than 45% in 60 minutes, using the USP Dissolution Method I, measured in a dissolution medium at pH 1.0 or at pH 6.1, both of which are under 50 rpm.

In another preferred embodiment, wherein the dosage is in the form of suspension, based on weight calculation, the formulation comprises celecoxib at 0.5-5% (w/v), more preferably 0.5-3% (w/v), and even more preferably 1-2% (w/v).

In another preferred embodiment, wherein the dosage is in the form of suspension, based on weight calculation, the formulation comprises sodium dodecyl sulfate at 0.1-2% (w/v), more preferably 0.1-1.5% (w/v), and even more preferably 0.1-1% (w/v). In another preferred embodiment, wherein the dosage is in the form of suspension, based on the weight calculation, the formulation comprises polyvinylpyrrolidone at 0.05-2% (w/v), more preferably 0.05-1% (w/v), and even more preferably 0.05-0.5% (w/v).

In another preferred embodiment, wherein the dosage is in the form of suspension, based on weight calculation, the formulation comprises sucrose at 0.5-30% (w/v), more preferably 0.5-20% (w/v), and even more preferably 0.5-10% (w/v).

In the second aspect of the disclosure, there is provided a manufacturing process for preparing the celecoxib oral formulation, wherein the dosage is in the form of tablets, capsules or granules. The said process comprises the following procedures:

Step A: prepare celecoxib into a nanoparticulate dispersion by an all-aqueous wet-milling process, and wherein the dispersion contains sodium dodecyl sulfate as surfactant polyvinylpyrrolidone as hydrophilic polymer.

Step B: add saccharides to the nanoparticulate dispersion obtained from Step A; add the remaining sodium dodecyl sulfate, and polyvinylpyrrolidone; mix the dispersion to obtain a homogeneous one; add one or two or more saccharides selected from a group consisting of monosaccharides, disaccharides, and polyols; preferably one or two or more saccharides selected from the group consisting of lactose, sucrose, fructose, mannitol, and sorbitol; wherein mixing is conducted with mechanical stirring;

Step C: prepare drug-loaded particles or drug-loaded spheres using a fluidized-bed drying process by spraying the nanoparticulate dispersion obtained from Step B onto the carriers;

Step D: prepare an oral solid dosage form such as tablets, capsules, or granules, using the above drug-loaded particles or drug-loaded spheres or drug-loaded powder.

In another preferred embodiment, for the wet-milling in Step A, based on weight calculation, the nanoparticulate dispersion comprises celecoxib at more than 10%, more preferably 10-35% w/w, more preferably 15-25% w/w.

In another preferred embodiment, based on weight calculation, the nanoparticulate dispersion obtained in Step B comprises sodium dodecyl sulfate at 0.5-12% w/w, more preferably 2-10% w/w, and even more preferably 4-8% w/w; it also comprises polyvinylpyrrolidone at 0.5-7% w/w, more preferably 0.5-5% w/w, and even more preferably 0.5-3% w/w; it also comprises saccharides at 10-70%, more preferably 10-50% w/w, and even more preferably 10-30% w/w.

In another preferred embodiment, the carriers used in Step C include filler or sphere, wherein the filler comprises one or two or more saccharides selected from a group consisting of monosaccharides, disaccharides, and polyols, and the sphere is selected from a group consisting of sucrose spheres, microcrystalline cellulose spheres, starch spheres, lactose spheres, silicon dioxide spheres, hypromellose spheres, citric acid spheres. or tartaric acid spheres.

In another preferred embodiment, the carrier further comprises one or two or more excipients selected from the group consisting of disintegrant, binder, glidant, lubricant, and antioxidant.

When the dosage is the form of suspension, the preparation includes Step A, Step B, and Step E as follows:

Step A: prepare celecoxib into a nanoparticulate dispersion by an all-aqueous wet-milling process; wherein sodium dodecyl sulfate is used as a surfactant, and polyvinylpyrrolidone is used as a hydrophilic polymer;

Step B: add saccharides to the nanoparticulate dispersion obtained from Step A; continue to add sodium dodecyl sulfate and polyvinylpyrrolidone, and mix the dispersion to obtain a homogeneous one; wherein one or two or more saccharides used are selected from a group consisting of monosaccharides, disaccharides, and polyols; preferably one or two or more saccharides used are selected from the group consisting of lactose, sucrose, fructose, mannitol, and sorbitol; and wherein preferably mixing is carried out by stirring; and

Step E: add one or two or more of the following excipients to the nanoparticulate dispersion obtained in Step B: suspending agents, antioxidants, taste masking agents, sweeteners, preservatives, defoamers, thickeners, fragrants, pH buffering salts; preferably mixing is carried out by stirring.

In the third aspect of the present disclosure, there is provided the use of celecoxib oral formulation as described above for the treatment of mild to moderate acute pains, and/or mild to moderate chronic pains, or the manufacturing method of the celecoxib formulation for the treatment of mild to moderate acute pains and/or mild to moderate chronic pains.

### BRIEF DESCRIPTION OF THE FIGURES

The present disclosure will be further explained with the accompanying figures. These are not necessarily to scale, and the purpose of which is to illustrate key points of the disclosure. In addition, part of the figures may be enlarged to highlight certain features of the disclosure.

FIG. 1 shows the chemical structure of celecoxib.

FIG. 2A shows the particle size distribution of a representative batch of celecoxib (pre-mill); FIG. 2B shows the particle size distribution of celecoxib (after-mill) in a representative batch of nanoparticulate dispersion of an embodiment in the disclosure.

FIG. 3 shows the manufacturing process of celecoxib formulation, including oral solid dosage forms such as tablets, capsules and granules, and oral liquid dosage forms such as suspension.

FIG. 4 shows the mean plasma concentration measured on a 0-12 hour scale after single oral dosing to male and female beagle dogs with using an embodiment in the present disclosure, including Celecoxib Test Formulation 1 (50 mg/capsule), Celecoxib Test Formulation 2 (25 mg/capsule), and the commercial celecoxib product Celebrex® (100 mg/capsule).

FIGS. 5A and 5B show the mean plasma concentration vs. time for human subjects after oral administration of celecoxib formulation disclosed in the present invention. In the study, 12 healthy volunteers were administered separately in 4 Treatments A, B, C, and D, under fasting conditions; wherein Treatment A: celecoxib formulation AP2500 50 mg/capsule; Treatment B: Celebrex °, a commercial product of celecoxib, 100 mg/capsule; Treatment C: celecoxib formulation AP2500 75 mg/capsule; Treatment D: celecoxib formulation AP2500, 100 mg/capsule. FIG. 5A shows the mean plasma concentration vs. time for celecoxib over 0-48 hours. FIG. 5B shows the mean plasma concentration vs time for celecoxib over 0-12 hours.

The above figures constitute a part of this specification and include illustrative embodiments of the present invention and illustrate various objects and features thereof. Further, the figures are not necessarily to scale; some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### DETAILED DESCRIPTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiments, though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, unless the context indicates otherwise, the term “or” used in the present disclosure is inclusive and equivalent to the term “and/or”.

### Definition

The term “AUC” or “area under the curve” used in the disclosure refers to the integration of the area under the curve of plasma concentration versus time or the drug-time curve after administration, reflecting the degree of absorption of the drug in humans.

The term “Cₘₐₓ” (peak concentration) used in the present disclosure refers to the peak concentration of a pharmaceutical ingredient in plasma after administration. The term “Tₘₐₓ” (time to peak concentration) used in the present disclosure refers to the time when the drug in the plasma reaches the highest concentration after administration.

The term “bioavailability” in the present disclosure refers to the degree to which a drug is utilized in a biological system.

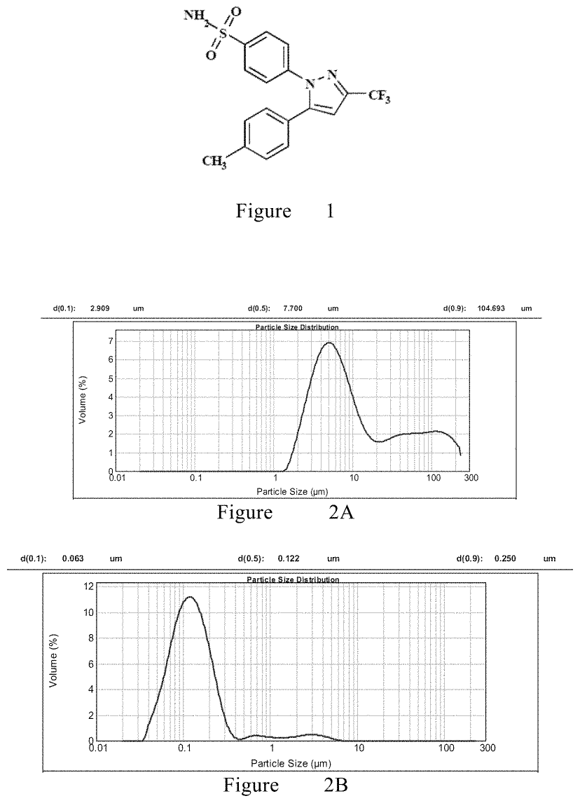

The term “celecoxib” as used herein refers to the compound 445-(4-methylphenyl)-3-(trifluoromethyl)-1hydro-pyrazol-1-yl] benzenesulfonamide, having a chemical structure shown in FIG. 1. Celecoxib has a molecular weight of 381.4 and is a white crystalline powder with a melting point of 157-158° C. Celecoxib is a selective cyclooxygenase-2 (“COX-2”) inhibitor to be used in the treatment of osteoarthritis (OA), rheumatoid arthritis (RA), some acute pains, menstrual pains, etc.

The term “reference” used in the present disclosure refers to Celebrex® capsules having the following strengths: 50 mg, 100 mg, 200 mg or 400 mg.

As used in the present disclosure, the term “celecoxib formulation” or “celecoxib composition” or “composition” or “celecoxib test formulation” or “low-strength celecoxib formulation” or “low-dose celecoxib formulation” or “AP2500” all refer to the celecoxib formulations in the present disclosure. Celecoxib formulations include oral liquid dosage forms such as suspension and oral solid dosage forms such as capsules, tablets, granules, etc. AP2500 specifically refers to an oral capsule formulation of celecoxib.

The term “commercial product” or “commercial celecoxib product” used in the present disclosure refers to Celebrex® or a generic product with the equivalent strength and bioequivalence as Celebrex, the strengths of the products are: 50 mg, 100 mg, 200 mg or 400 mg; the dosage is in the form of capsules or tablets.

The term “bioequivalent” as used in the present disclosure means that, as compared with the commercial product, the C. of the low-dose celecoxib formulation of the disclosure in humans is at the same level (i.e., in the 90% confidence interval, with the geometric mean ratio of C. being in the range of 80%-125%), or AUC is at the same level (i.e., in the 90% confidence interval, with the geometric mean ratio of AUC being in the range of 80%-125%), or both C. and AUC of the celecoxib formulation in the body are at the same level (i.e., in the 90% confidence interval, with the geometric mean ratio of C. and AUC being in the range of 80%-125%).

The term “confidence interval” used in the present disclosure refers to the estimated interval of the overall parameter constructed by the sample statistics. In statistics, the confidence interval (“CV”) of a probability sample is an interval estimate of a certain overall parameter of the sample. The confidence interval shows the degree to which the true value of this parameter has a certain probability to fall around the measurement result. The confidence interval gives the credibility of the measured value of the measured parameter, that is, the “a probability” required above.

The term “dissolution” as used in the disclosure refers to the process of a drug releasing from a dosage form such as a capsule or a tablet. “Dissolution” or “dissolution rate” refers to the rate and degree of dissolution of a drug released from a solid dosage form such as tablets or capsules in a preset dissolution media.

The “dose” used in the present disclosure refers to the amount that achieves a therapeutic effect of a drug after administration. The “effective dose” as used in the present disclosure refers to the dose of a drug that achieves a therapeutic effect in the body through a specific route of administration.

The term “strength” used in the present disclosure refers to the total amount of the drug contained in a dosage form, such as a capsule, a tablet or a suspension of a unit volume.

The term “non-steroidal anti-inflammatory drugs” or “NSAIDs” used in the disclosure refers to a class of drugs that do not contain steroidal-type chemical structure but have anti-inflammatory, antipyretic and analgesic effects. Hence, they are known as non-steroidal anti-inflammatory drugs. Non-steroidal anti-inflammatory drugs are the first-line drugs for the treatment of osteoarthritis. They are also widely used in other bone and joint diseases, rheumatic immune diseases and pains of all sorts, in order to alleviate the pain and stiffness of the above-mentioned diseases and to improve the function of bones and joints. Celecoxib is one of the NSAIDs, which can be administered orally to patients. It can also be used for acute pains.

The term “acute pain” refers to the pain that comes on quickly, sometimes even intense, but only lasts a relatively short period of time. As used herein, “mild to moderate acute pain” refers to a commonly used terminology, which quantifies the degree of pain when one measures his/her pain as less than 7 on a scale of zero-to-ten. Mild to moderate acute pain may include back pain, neck pain, migraine, pain after small surgery, etc. As used herein, “chronic pain” is used as opposed to “acute pain”. “Chronic pain” usually refers to the pain that lasts longer than 6 months. Chronic pain can be mild or excruciating, episodic or continuous, merely inconvenient or totally incapacitating. The most common sources of pain stem from headaches, joint pain, pain from injury, and backaches. Other kinds of chronic pain include tendinitis, sinus pain, carpal tunnel syndrome, and pain affecting specific parts of the body, such as shoulders, pelvis, and neck. Generalized muscle or nerve pain can also develop into a chronic condition.

The terms “subject(s)” or “individual(s)” or “patient(s)” used in the present disclosure are used interchangeably and refer to mammals (including humans).

The term “non-naive” used in the present disclosure refers to animals that have been previously treated in other experiments, such as pharmacokinetic studies. In the present disclosure, the term “non-compartmental model” refers to a commonly used mathematical modeling method in pharmacokinetic studies.

The term “particle size” used in the present disclosure is to describe an important biophysical property of the drug nanoparticles, i.e., the size and the distribution of the particles. The particle size and stability directly affect the absorption, distribution, efficacy and safety of nanoparticle-based pharmaceutical products in vivo. Parameters of the particle size distribution, such as D50 and D90, also known as d(0.5) and d(0.9), can be measured by a laser particle size analyzer. The D50 refers to the particle size value when the cumulative particle size distribution percentage reaches 50%, and D50 value is usually used in place of the average particle size. Similarly, the D90 refers to the particle size value when the cumulative particle size distribution percentage reaches 90%.

In the present disclosure, “stability” refers to the physical and chemical stability of the formulation in a defined testing condition. The storage condition of 40° C./75% RH in the disclosure is a commonly used accelerated stability condition, which is intended to assess both physical and chemical stability of the celecoxib formulation. Storage conditions generally comes with a “time-period” (e.g., 2 weeks, 1 month, 3 months), indicating the actual time that the formulation samples have undergone in such specified storage condition.

The term “pharmaceutically acceptable excipients” or “excipients” in the disclosure refer to those excipients and adjuvants used in formulations. They have been reasonably evaluated in terms of safety and are included in pharmaceutical formulations. There are different categories of pharmaceutically acceptable excipients, with different functions. For example, for oral solid formulations, pharmaceutically acceptable excipients include categories such as: fillers, binders, disintegrants, glidants, antioxidants, and the like. For oral suspensions, pharmaceutically acceptable excipients include: suspending agents, antioxidants, taste masking agents, sweeteners, preservatives, antifoaming agents, thickeners, pH buffering agents, and the like. The pharmaceutical excipients are important components in a formulation. The selection and usage of these excipients directly impact the safety and effectiveness of the drug, as well as product stability.

The term “wet-milling” used in the disclosure refers to a process for preparing drug nanoparticulate dispersions: into the milling chamber of the equipment, add grinding media (or “grinding beads”), then add the dispersion that contains the drug, surfactant(s), hydrophilic polymer(s) and other pharmaceutically acceptable excipients. This is followed by initiating high-speed rotation of the stirring rod in the chamber, which allows the grinding beads to impact, shear, and grind the drug particles in the aqueous dispersion, so as to reduce the drug particle size from the micron meter range to nanometer range (usually 20-1000 nm).

The term “carrier(s)” used in the present disclosure specifically refers to pharmaceutically acceptable excipients placed at the chamber bottom of the fluidized-bed dryer.

Low-strength (i.e., low-dose) celecoxib oral formulations The present disclosure provides a low-strength (i.e., low-dose) celecoxib oral formulation that can be used to treat pain and inflammation, so as to achieve the purpose of safer use.

In some embodiments, the present disclosure provides a celecoxib formulation in a dosage of, for example, capsules, tablets, granules or suspensions. In a pharmacokinetic study on healthy volunteers under fast condition, compared with the commercial celecoxib product Celebrex, the peak plasma concentration C. of the formulation of the present disclosure can reach a bioequivalence, within the 90% confidence interval, and the geometric mean ratio of C. is 80%-125%; or the AUC can reach a bioequivalence, in the 90% confidence interval, and the geometric mean ratio of AUC is 80%-125%; or Cₘₐₓ and AUC are both bioequivalent, in the 90% confidence interval, and the geometric mean ratios of both C. and AUC are 80%-125%. In the meantime, the drug dose of the celecoxib formulation of the present disclosure is reduced by 10-40% w/w compared with the commercial celecoxib product Celebrex of the corresponding strength.

The strengths of commercial celecoxib product Celebrex or its generics are: 50 mg, 100 mg, 200 mg, 400 mg. With respect to the above four strengths, the new strengths of the celecoxib oral solid formulations of the present disclosure in the dosage of, for example, tablets, capsules or granules, are reduced by 10-40%. For example, corresponding to a commercial 50 mg product, the new strength can be: 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg. For example, corresponding to a commercial 100 mg product, the new strength can be: 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg. For example, corresponding to a commercial 200 mg product, the new strength can be: 120 mg, 122 mg, 124 mg, 126 mg, 128 mg, 130 mg, 132 mg, 134 mg, 136 mg, 138 mg, 140 mg, 142 mg, 144 mg, 146 mg, 148 mg, 150 mg, 152 mg, 154 mg, 156 mg, 158 mg, 160 mg, 162 mg, 164 mg, 166 mg, 168 mg, 170 mg, 172 mg, 174 mg, 176 mg, 178 mg, 180 mg. For example, corresponding to a commercial 400 mg product, the new strength can be: 240 mg, 244 mg, 248 mg, 252 mg, 256 mg, 260 mg, 264 mg, 268 mg, 272 mg, 276 mg, 280 mg, 284 mg, 288 mg, 292 mg, 296 mg, 300 mg, 304 mg, 308 mg, 312 mg, 316 mg, 320 mg, 324 mg, 328 mg, 332 mg, 336 mg, 340 mg, 344 mg, 348 mg, 352 mg, 356 mg, 360 mg.

In some embodiments, when the strengths of the celecoxib oral solid formulations of the present disclosure are reduced by 20%, i.e., 40 mg, 80 mg, 160 mg, and 320 mg; they respectively correspond to the strengths of the commercial products of 50 mg, 100 mg, 200 mg, 400 mg.

In some embodiments, the celecoxib oral solid formulation may be in a dosage such as tablets, capsules, granules, orally disintegrating tablets, and sublingual tablets.

In some embodiments, the celecoxib formulation is in the dosage form of an oral suspension.

In some embodiments, the strength of the celecoxib formulation are 40 mg and 80 mg as capsules, respectively. Using the USP Dissolution Method I (Apparatus I, basket, 50 rpm), when at pH 1.0, the dissoluted amount of celecoxib is not less than 30% in 30 minutes and not less than 45% in 60 minutes.

In some embodiments, the strengths of the celecoxib formulation are 40 mg and 80 mg as capsules, respectively. Using the USP Dissolution Method I (Apparatus I, basket, 50 rpm), when at pH 6.1, the dissoluted amount of celecoxib is not less than 30% in 30 minutes and not less than 45% in 60 minutes.

### Preparation of Celecoxib Oral Formulations

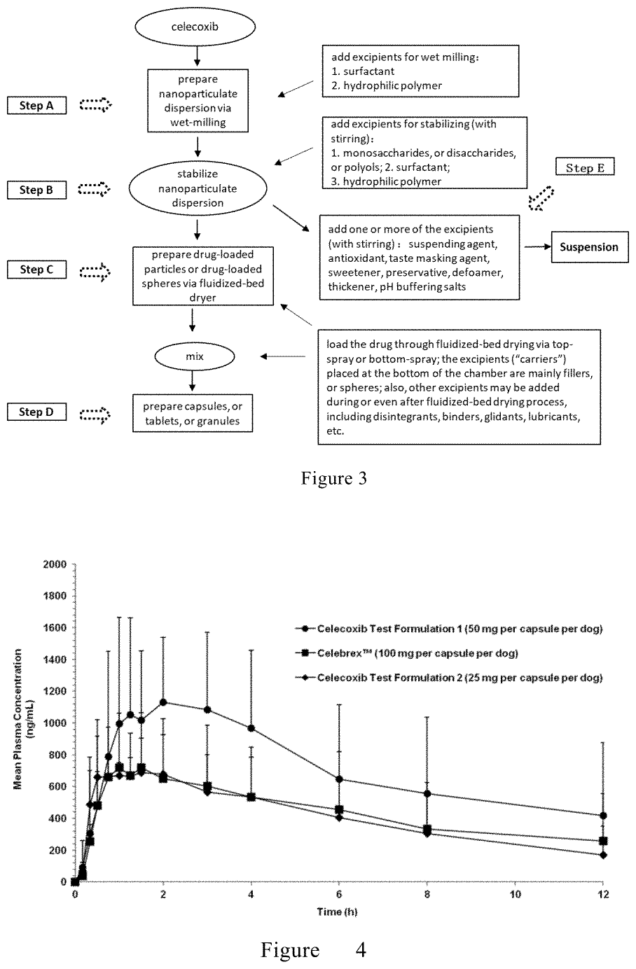

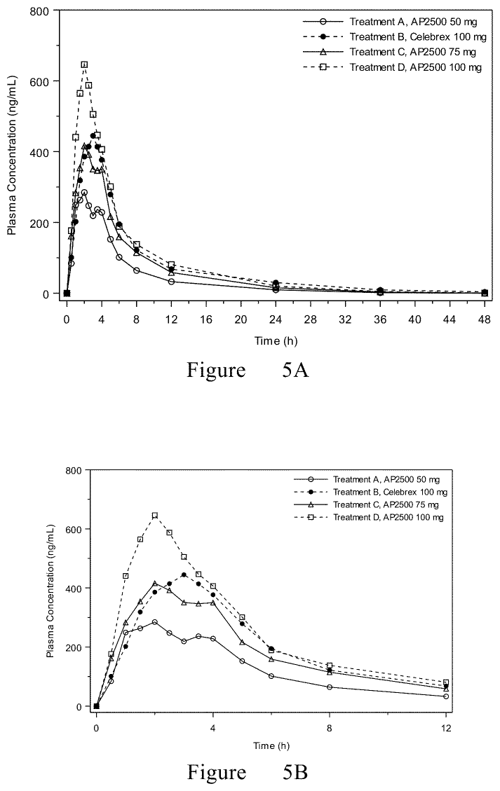

In some embodiments, the preparation of the celecoxib oral formulations of the present disclosure involves the following procedures (see FIG. 3):

Step A: prepare nanoparticulate dispersion by wet-milling.

Step B: stabilize the nanoparticulate dispersion by adding saccharides and other excipients.

Step C: prepare drug-loaded particles or drug-loaded spheres or drug-loaded powders, using fluidized-bed drying process while removing water.

Step D: prepare celecoxib solid formulations.

The preparation for celecoxib nanoparticulate dispersion in the present disclosure utilizes a two-step procedure: “Step A, then Step B”, or “mill, then stabilize”.

Step A: prepare a nanoparticulate dispersion by wet-milling: add celecoxib, surfactant(s), and hydrophilic polymer(s) to the aqueous phase to form a dispersion; then initiate the high-speed rotation of the stirring rod (2600-4500 rpm), which allows the grinding media (“grinding beads”) in the milling chamber to impact, shear, and grind the drug particles in dispersion, thereby reducing the drug particles from micron-meter ranges (e.g., 20-200 μm) to nano-meter ranges (e.g., 20-400 nm).

The wet-milling process has certain requirement for surfactant(s) and hydrophilic polymer(s) with regard to the screening, the amount and usage, which is a key to ensure that the particle size of celecoxib can be milled to the nano-meter range. During milling process, the drug particles gradually decrease in size, while the surface area of these particles increases dramatically. The volume of the milling chamber depends on the manufacturing scale. Usually it is as follows: 0.2L, 0.5L, 1L, 2L, 5L, and 10L. The milling chamber is usually connected externally to a container that is 2-20 times greater in volume than the milling chamber itself, which can be used for various manufacturing needs from lab testing, pilot scales, and all the way to large-scale production. The grinding beads are held in the milling chamber by a dynamic gap separator. These are usually the zirconia beads stabilized with yttria (YTZ) with the bead diameter of 0.1-0.5 mm, or beads made of polystyrene spherical resin with a high degree of cross-linking of the polymers. The temperature in milling chamber is usually controlled at 20-45° C. In the present disclosure, the milling for preparing nanoparticulate dispersion is an all-aqueous phase which does not involve organic solvent. The present disclosure also does not include any high-pressure homogenization procedure.

The wet-milling process of celecoxib needs to evaluate a number of technical parameters in order to optimize the composition and the process, and to obtain a celecoxib nanoparticulate dispersion with acceptable physical and chemical properties and especially a stabilized drug particle size. Some of the important technical parameters in milling include: drug loading, the category and the concentration of the excipients to be used, the amount as well as the size of the grinding beads, milling speed, milling time, chamber temperature, feeding rate from peristaltic pump, and the like.

Step B: add excipients into the nanoparticulate dispersion obtained from the above-described wet-milling process (Step A), with mechanical stirring, so as to stabilize the drug particle size in such dispersion. The added excipients include some of the following excipients: monosaccharides, disaccharides, or polyols and other highly-hydrophilic excipients, while in the same time, continue to add more of surfactants and hydrophilic polymers.

Wet-milling, as described in Step A, is a process wherein the aqueous phase comprising surfactant(s) and hydrophilic polymer(s), the grinding rod is rotated in the milling chamber to drive the milling beads, and through high-speed impact, shearing, grinding and the like, to reduce the drug particles from micron-meter range to the nano-meter range. After which, prepare the drug-loaded solid particles through fluidized-bed drying process or other drying and de-watering processes, followed by mixing the solid particles with other pharmaceutically acceptable excipients. The nanoparticle-based drug solids were thus prepared. The drugs through wet-milling must meet the following requirement:

1) drug particle size is reduced to the nanometer range. For example: D50 is less than 200 nm; D90 is less than 400 nm; or preferably, D50 is less than 150 nm, and D90 is less than 300 nm;

2) during milling, if the drug loading is high in dispersion, there would be a greater milling efficiency. Normally the drug loading is greater than 5% w/w, greater than 10% w/w; for drugs with high strengths (e.g., 50 mg or more), the drug loading in dispersion is expected to be in the range of 15-25% w/w;

3) the nanoparticulate dispersion needs to be sufficiently stable to ensure an operatable process. During large-scale manufacturing, there usually exists time-lag between wet-milling and subsequent process—which requires the nanoparticulate dispersion of certain level of stability, such as 48-72 hours, preferably 7 days, i.e., the chemical and physical properties of the drug particles and especially the drug particle size remain no appreciable difference (less than 10%, or ideally less than 5%).

4) the drug particle size needs to remain stable, i.e., within certain nanometer range (20-400 nm), during the process of de-watering and forming drug-loaded particles or drug-loaded spheres or drug-loaded powder. This is critical: in the process of manufacturing nanoparticle-based dosages such as tablets, capsules or granules, when water is removed, and the drug particles is solidified onto the surface of the excipients (also known as “carrier” in fluidized-bed drying process), the drug particles need to remain as in nano range and as stable. For only a nanoparticle-based and stable formulation can ensure that when the drug is released in the body, it accomplishes what it was set to accomplish as nanoparticles: fast dissolution, and high absorption.

In the present disclosure, the preparation of celecoxib nanoparticulate dispersion takes an approach as “mill, then stabilize”, i.e., “Step A, then Step B”, which effectively tackles several key issues arising from the wet-milling of celecoxib:

1) it increases the celecoxib loading in the milling dispersion (from 10% w/w to 20%-25% w/w). This is important for large-scale celecoxib milling process (or commercial production). Previously, for milling celecoxib, when loading is higher than 10% w/w, there will be fast growing of foaming caused by the surfactant, the increasing of dispersion viscosity, as well as the temperature increment inside the milling chamber (up to 50° C.−65° C.), which makes the milling process difficult and sometimes even impossible. However, when only a portion of the surfactant and a portion of the hydrophilic polymer are used during milling, the foaming and the viscosity issues would substantially alleviate, and the manufacturing process becomes manageable. In the meantime, the temperature inside the milling chamber maintains within an operatable range (15° C.−40° C.). By doing this, one can raise the celecoxib loading milling dispersion to 20%-30% w/w, which greatly enhances milling efficiency, and celecoxib reaches expected nanometer range (i.e., D50 less than 160 nm, D90 less than 300 nm) in a relatively short milling time (e.g., 2-4 hours);

2) it effectively improves the particle size stability for celecoxib to stay within nanometer range. When celecoxib completes wet-milling after it reaches the preset requirement for particle size, the dispersion is taken out and is added with excipients such as saccharides and the remaining portions of the surfactant and the hydrophilic polymer. The addition of these excipients is carried out with conventional mechanical stirring. These added excipients effectively enhance celecoxib particle size stability in nanoparticulate dispersion over the processing time. In particular, the application of excipients such as saccharides or polyols (such as lactose, mannitol or sucrose) significantly improves celecoxib particle size stability within nanometer range in subsequent fluidized-bed drying process for water removing (i.e., the drug loading and the solidification upon mixing with other excipients under hot air flow during fluidized-bed drying). This is substantiated in later dissolution and stability studies for celecoxib formulations.

Step C: prepare the drug-loaded particles or drug-loaded spheres or drug-loaded powder while removing water and mixing with other pharmaceutical excipients. A process such as fluidized-bed drying is commonly used water removing and granulation. The two approaches are:

1) prepare drug-loaded particles or powder: via top-spray or bottom-spray. The procedures are as such: spray the celecoxib nanoparticulate dispersion onto the excipients placed beforehand at the chamber bottom of the fluidized-bed dryer (excipients placed at the bottom of the chamber are also called “carriers”). The nanoparticulate dispersion and the carrier are vigorously and continuously mixed, tumbled, de-watered, and dried up inside the chamber with the heated air flowing out from the bottom, and finally the nanoparticles are attached onto the surface of the excipients, forming dried drug-loaded particles or powder.

2) prepare drug-loaded spheres: via bottom-spray. The procedures are as such: spray the celecoxib nanoparticulate dispersion onto the spheres placed beforehand at the chamber bottom of the fluidized-bed dryer. Similarly, the nanoparticulate dispersion and the carrier are vigorously and continuously mixed, tumbled, de-watered, and dried up inside the chamber with the heated air flowing out from the bottom, and finally the nanoparticles are attached to the surface of the excipients, forming a dried drug-loaded spheres.

The stability of the celecoxib nanoparticle-based formulation and the flowability of the granules are directly related to the following factors: the category and the amount of the excipients used, the ratio between the drug and the excipients, and actual equipment for fluidized-bed drying (chamber volume, nozzle diameter), operational parameters such as inlet air temperature, air volume, material temperature inside chamber, atomization pressure, spray rate, drying time, etc.

In the fluidized-bed drying process for preparing drug-loaded particles or drug-loaded spheres, it is necessary to add certain excipients in the nanoparticulate dispersion for subsequent spraying. It is also necessary to screen and optimize the carriers (i.e., excipients or spheres to be placed at the chamber bottom of the fluidized-bed). These contribute not only to the nanoparticle stability upon solidifying onto the excipients or spheres, but also to the flowability of the drug-loaded particle powders or drug-loaded spheres in subsequent preparation of capsules or tablets or granules and the like. The carriers (excipients) used include but not are not limited to, monosaccharides (such as glucose, fructose, galactose, ribose, deoxyribose), disaccharides (such as sucrose, maltose, lactose), or polyols (such as mannitol, xylitol, sorbitol), etc. Based on total weight calculation of drug-loaded particles or drug-loaded spheres through fluidized-bed drying process, the content of the saccharide is in the range of 30-70% w/w. The carriers (excipients) can also be spheres, including but not limited to: sucrose spheres, microcrystalline cellulose spheres, starch spheres, tartaric acid spheres, lactose spheres, etc. Based on total weight calculation, the drug-loaded particles which obtained through fluidized-bed drying process, has a content of spheres at a range of 30-70% w/w.

Besides fluidized-bed drying process, there are other manufacturing processes can be used in Step C in removing water and granulation, including: high-efficiency coating and drying, vacuum drying, box drying, and spray coating drying, etc.

Step D: prepare celecoxib formulations: upon completing fluidized-bed drying and granulation, the celecoxib-loaded powder or celecoxib-loaded spheres have reasonably good flowability in general and can be used straightforward to prepare capsules, tablets or granules. The celecoxib-loaded powder or celecoxib-loaded spheres can be mixed to the fullest extent with other excipients, including disintegrants, and/or binders, and/or glidants, and/or lubricants, etc., during Step C or afterwards, and then filled into capsules, or compressed into tablets, or made into granules according to varying manufacturing requirements.

In some embodiments, the surfactants used in the wet-milling of celecoxib (Step A) and the subsequent preparation of stable nanoparticulate dispersion (Step B) include nonionic surfactants, cationic surfactants, anionic surfactants, zwitterionic surfactants, including but not limited to: sodium dodecyl sulfate, sodium stearyl sulfate, sodium hexadecyl sulfate, sodium stearyl sulfate, dioctyl sodium sulfosuccinate (DOSS), sodium deoxycholate, monooleate, monolaurate, monopalmitate, monostearate or polyoxyethylene sorbitan, phospholipid, poloxamer 188, poloxamer 338, poloxamer 407, cholic acid, sodium cholate, deoxycholic acid, sodium taurocholate, taurocholic acid, taurodeoxycholate, taurodeoxycholic acid, soy lecithin, casein, phospholipids, poloxamer, poloxamine, polysorbate 20, polysorbate 40, polysorbate 60, polysorbate 80. Upon completing Steps A and B, the total content of the surfactant in the dispersion is at 0.5%-12% w/w, preferably 2-10% w/w, more preferably 4-8% w/w.

In some embodiments, the surfactants used in the wet-milling of celecoxib (Step A) and subsequent preparation of a stable nanoparticulate dispersion (Step B) include sodium dodecyl sulfate, the total content of which in the dispersion (i.e., Step A, then Step B) is at 4-8% w/w.

In some embodiments, the hydrophilic polymers used for wet-milling (Step A) and subsequent preparation of stable nanoparticulate dispersions (Step B) include polyvinylpyrrolidone (PVP), polyvinylpyrrolidone/vinyl acetate (PVP/VA) copolymer (such as PVP/VA 64, PVP/VA 37), hydroxymethyl cellulose (HMC), hydroxyethyl cellulose (HEC), hydroxypropyl methyl cellulose (HPMC), hydroxypropyl cellulose (HPC), methyl cellulose (MC), ethyl cellulose (EC), hydroxymethyl ethyl cellulose (HEMC), ethyl hydroxyethyl cellulose (EHEC) and carboxymethyl cellulose, polyethylene glycol 4000, polyethylene glycol 6000, polyethylene glycol 8000, polyethylene glycol 10000, polyethylene glycol 20000. Upon completing Steps A and B, the total content of the hydrophilic polymer in the celecoxib dispersion is at 0.5%-7% w/w, preferably 0.5-5% w/w, more preferably 0.5-3% w/w.

In some embodiments, the hydrophilic polymer used in the wet-milling of celecoxib (Step A) and subsequent preparation of a stable nanoparticulate dispersion (Step B) includes polyvinylpyrrolidone; the total content of polyvinylpyrrolidone in the celecoxib dispersion (i.e., Step A, then Step B) is at 0.5-3% w/w.

In some embodiments, the saccharides used for preparing a stable celecoxib nanoparticulate dispersion (Step B) include monosaccharides (such as glucose, fructose, galactose, ribose, deoxyribose), disaccharides (such as: sucrose, maltose, lactose), or polyols (such as: mannitol, xylitol, sorbitol) and other highly hydrophilic excipients; the content of saccharides in the celecoxib dispersion is at 10-60% w/w, preferably 10-40% w/w, more preferably 10-30% w/w.

In some embodiments, the saccharides used in Step B for preparing a stable celecoxib nanoparticulate dispersion is sucrose, and the total content of sucrose in the celecoxib dispersion is at 10-30% w/w.

In some embodiments, the pharmaceutically acceptable excipients used in fluidized-bed drying for preparing celecoxib's loaded particles or loaded spheres or loaded granules (also known as “carrier” which is placed at the bottom of the fluidized-bed; Step C) include one or more excipients from a group consisting of fillers, binders, disintegrants, lubricant, glidant, antioxidant, or any combination of these excipients. The total content of these excipients in celecoxib formulations is at 5%-80% w/w.

In some embodiments, the fillers used for the fluidized-bed drying and subsequent mixing include, but are not limited to: monosaccharides (such as glucose, fructose, galactose, ribose, deoxyribose), disaccharides (such as sucrose, maltose, lactose), polyols (such as mannitol, xylitol, sorbitol); starch, cellulose (microcrystalline cellulose, methyl cellulose, carboxymethyl cellulose, hydroxypropyl methyl cellulose, hydroxypropyl cellulose, hydroxyethyl cellulose), silicified microcrystalline cellulose (“SMCC”), anhydrous calcium phosphate or calcium dihydrogen phosphate, calcium carbonate, calcium sulfate; In some embodiments, spheres used include: sucrose spheres, microcrystalline spheres starch spheres, lactose spheres, silicon dioxide spheres, hypromellose spheres, citric acid spheres, tartaric acid spheres. The above-mentioned spheres are of various diameters: 0.212-0.355 mm; 0.3-0.5 mm; 0.3-0.425 mm; 0.425-0.5 mm; 0.425-0.6 mm;0.5-0.6 mm; 0.5-0.71 mm; 0.6-0.71 mm; 0.71-0.85 mm; 0.71-0.9 mm;0.8-0.9 mm; 0.85-1.0 mm; 0.9-1.12 mm; 1.0-1.18 mm. The total content of fillers in celecoxib formulations is at 20-80% w/w.

In some embodiments, the binders used for celecoxib in a fluidized-bed drying and subsequent mixing include, but are not limited to: gum arabic, gelatin, polymethacrylate, polyvinylpyrrolidone, starch, pregelatinized starch, tragacanth gum, xanthan gum, alginate, magnesium-aluminum silicate, bentonite, etc.

In some embodiments, the disintegrants used for celecoxib in a fluidized-bed drying process and subsequent mixing include, but are not limited to: starch, pregelatinized starch, hydroxypropyl starch, sodium carboxymethyl starch, sodium carboxymethyl cellulose, cross-linked sodium carboxymethyl cellulose, cross-linked polyvinylpyrrolidone.

In some embodiments, the lubricants and/or glidants used for celecoxib in a fluidized-bed drying process and subsequent mixing include, but are not limited to: magnesium stearate, calcium stearate, talc, polyethylene glycol, ethylene oxide polymer, sodium dodecyl sulfate, magnesium dodecyl sulfate, sodium oleate, sodium stearate fumarate, stearic acid, silica, micronized silica gel, silica gel.

In some embodiments, the antioxidants used for celecoxib in a fluidized-bed drying process and subsequent mixing include, but are not limited to: free radical absorbers (such as vitamin E, carotenoids), oxygen scavengers (such as carotenoids and their derivatives, ascorbic acid, ascorbic palmitate, erythorbic acid, sodium erythorbate), metal ion chelating agents (such as citric acid, EDTA, phosphoric acid derivatives), butyl hydroxyanisole (BHA), dibutyl hydroxytoluene (BHT).

In some embodiments, the celecoxib formulation contains sucrose; its content in the oral formulation is at 10%-80% w/w, preferably 20-70% w/w, more preferably 30-70% w/w.

In some embodiments, the celecoxib formulation contains a sucrose spheres; the content of the spheres in the formulation is at 10%-80% w/w, preferably 20-70% w/w, more preferably 30-70% w/w.

In some embodiments, the celecoxib formulation contains microcrystalline cellulose spheres; its content in the formulation is at 10%-80% w/w, preferably 20-70% w/w, more preferably 30-70% w/w.

In one embodiment, the celecoxib formulation includes a moisture-proof film coating; the film coating material is usually composed of various celluloses such as hydroxypropyl methyl cellulose, ethyl cellulose, and titanium dioxide, talc, etc. Commonly used moisture-proof film coatings include: Opadry® II. Its content in the oral solid formulation is at 1-5% w/w.

In some embodiments, the celecoxib oral solid formulations is in the dosage forms of capsules, or tablets, or granules.

In one embodiment, a celecoxib formulation is prepared, with a strength as 25 mg per capsule; in one embodiment, a celecoxib formulation is prepared with a strength as 40 mg per capsule; in one embodiment, a celecoxib formulation is prepared with a strength as 50 mg per capsule; in one embodiment, a celecoxib formulation is prepared with a strength as 75 mg per capsule; in one embodiment, a celecoxib formulation is prepared with a strength as 80 mg per capsule; in one embodiment, a celecoxib formulation is prepared with a strength as 160 mg per capsule; in one embodiment, a celecoxib formulation is prepared with a strength as 320 mg per capsule.

In one embodiment, a celecoxib formulation is prepared with a strength as 25 mg per tablet; in one embodiment, a celecoxib formulation is prepared with a strength as 40 mg per tablet; in one embodiment, a celecoxib formulation is prepared with a strength as 50 mg per tablet; in one embodiment, a celecoxib formulation is prepared with a strength as 80 mg per tablet; in one embodiment, a celecoxib formulation is prepared with a strength as 80 mg per tablet; in one embodiment, a celecoxib formulation is prepared with a strength as 160 mg per tablet; in one embodiment, a celecoxib formulation is prepared with a strength as 320 mg per tablet.

In one embodiment, the preparation of celecoxib in a dosage form of suspension comprises the following steps (see FIG. 3): Step A: prepare a nanoparticulate dispersion by wet milling. This is the same Step A as described in the above for preparing oral solid formulation. That is, by adding celecoxib, surfactant, and hydrophilic polymer to form a dispersion, then starting the high-speed rotation of the stirring rod and allowing the grinding media in the milling chamber to impact, shear and grind the drug particles in dispersion, whereby reducing the drug particles from micron-meter range to nano-meter range (usually 100-300 nm). Step B: add excipients such as saccharides to prepare a stable nanoparticulate dispersion. This is the same Step B described above in preparing for oral solid formulation. That is, by adding a certain number of monosaccharides, disaccharides, or polyols and other highly hydrophilic excipients, as well as certain amount of surfactant and hydrophilic polymer to the obtained nanoparticulate dispersion prepared by wet milling from Step A. Preferably mechanical mixing is carried out in the process. Step E: upon completing Step B, continue to add one or two or more of the following excipients, including: suspending agent, antioxidant, taste masking agent, sweetener, preservative, defoaming agent, thickener, pH buffering salt in solution. The mixing is preferably carried out by mechanical stirring. Based on weight calculation, the suspension as a dosage form comprises celecoxib at 0.5-5% (w/v), preferably 0.5-3% (w/v), more preferably 1-2% (w/v); sodium dodecyl sulfate at 0.1-2% (w/v), preferably 0.1-1.5% (w/v), more preferably 0.1-1% (w/v); polyvinylpyrrolidone at 0.05-2% (w/v), preferably 0.05-1% (w/v), more preferably 0.05-0.5% (w/v); sucrose at 0.5-30% (w/v), preferably 0.5-20% (w/v), more preferably 0.5-10%(w/v). Upon completing the above Steps A, B, and E, it may still need certain necessary processes for oral suspension such as sterilization and bottling, in order to obtain celecoxib oral suspension.

The celecoxib oral formulation provided by the present disclosure can be used for the treatment of mild to moderate acute pains, mild to moderate chronic pains. The present disclosure provides with the manufacturing process for preparing such formulations for treating mild to moderate acute pains, and mild to moderate chronic pains.

In one embodiment, the subject is a mammal (beagle).

In one embodiment, the subject is a healthy human.

The present disclosure will be further explained below in conjunction with specific embodiments. It should be understood that these embodiments are only used to illustrate the present disclosure and not to limit the scope of the present disclosure. The experimental methods that do not indicate specific conditions in the following examples are usually consistent with conventional conditions or consistent with the conditions recommended by the manufacturer. Unless otherwise stated, all percentages, ratios, proportions, or parts are by weight. The unit of weight-volume percentage in the present disclosure is well-known to those skilled in the art, for example, which refers to the weight of the solute in a 100 ml solution. Unless otherwise defined, all professional and scientific terms used in the disclosure have the same meaning as those familiar to those skilled in the art. In addition, any method and material similar or equivalent to the content described can be applied to the method of the present disclosure. The preferred implementation methods and materials described in the disclosure are for illustration purposes only.

### Example 1

### Preparation of Celecoxib Nanoparticulate Dispersion Via Wet Milling

Table 1-1 presents a partial listing for screening celecoxib nanoparticulate dispersions via wet milling. The high-speed wet milling equipment were used in the disclosure: Dyno®-Mill ResearchLab, Dyno-Mill® MultiLab; technical parameters and scopes for milling were: milling chamber volume: 75-600 ml; agitator speed: 2600-4200 rpm; milling time: 1-3 hours; grinding beads: 0.3-0.5 mm YTZ grinding beads (yttria stabilized zirconia beads); feeding rate for dispersion into the milling chamber: 10±5ml/min. Measurement for particle size: Malvern2000 laser particle size detector was used to evaluate drug particle size distribution (D50, D90); Leica optical microscope was used to observe particle size dispersion. Table 1-1 show that: Dispersion 6 and Dispersion 7 had good particle size distribution. Note: the dispersion herein refers to an intermediate generated through wet milling, not the suspension as a dosage form.

FIGS. 2A-2B show the drug particle size distribution for a representative batch of celecoxib dispersion before and after milling.

FIG. 2A shows the particle size distribution for a representative batch of celecoxib before milling: D50: 7.7 μm, D90: 104.7 μm. FIG. 2B shows the particle size distribution for celecoxib as in “Dispersion 10” after milling: D50: 122 nm; D90: 250 nm. Note: the D50 is also known as d(0.5); and D90 as d(0.9).

TABLE 1-1
Screening Celecoxib Nanoparticulate Dispersions via Wet Milling

| Dispersion No. | Components* | % w/w | D50 (nm)/D90 (nm) |
|---|---|---|---|
| Dispersion 1 | celecoxib | 10 | 441/2753 |
| | SLS | 0.2 | |
| Dispersion 2 | celecoxib | 10 | 138/310 |
| | HPMC | 0.2 | |
| Dispersion 3 | celecoxib | 10 | 294/610 |
| | Poloxamer 188 | 0.2 | |
| Dispersion 4 | celecoxib | 10 | 254/458 |
| | DOSS | 0.2 | |
| Dispersion 5 | celecoxib | 10 | 1701/2844 |
| | PVP K30 | 0.2 | |
| Dispersion 6 | celecoxib | 10 | 130/261 |
| | SLS | 0.2 | |
| | HPMC | 0.2 | |
| Dispersion 7 | celecoxib | 10 | 128/260 |
| | SLS | 0.2 | |
| | PVP K30 | 0.2 | |
| Dispersion 8 | celecoxib | 10 | 129/346 |
| | SLS | 0.2 | |
| | Poloxamer 188 | 0.2 | |

*SLS: sodium dodecyl sulfate;
HPMC: hydroxypropyl methylcellulose;
PVP: polyvinylpyrrolidone;
DOSS: dioctyl sodium sulfosuccinate (a.k.a: docusate sodium);
Poloxamer: (ethylene oxide)-poly(propylene oxide) block copolymer.

TABLE 1-2
Stability Study of Celecoxib Nanoparticulate Dispersions

| | | | D50 (nm)/D90 (nm) 22-25° C. | | | | |
|---|---|---|---|---|---|---|---|
| Dispersion | com- | % | Day | Day | Day | Day | Day |
| No. | ponent | w/w | 1 | 3 | 7 | 15 | 60 |
| Dispersion | celecoxib | 20 | 150/ | Not detected | | | |
| 9 | SLS | 0.4 | 800 | | | | |
| | HPMC | 0.4 | | | | | |
| Dispersion | celecoxib | 20 | 122/ | 135/ | 139/ | 203/ | Not |
| 10 | SLS | 0.4 | 250 | 292 | 340 | 545 | detected |
| | PVP K30 | 0.4 | | | | | |
| Dispersion | celecoxib | 20 | 124/ | 125/ | 128/ | 130/ | 133/ |
| 11 | SLS | 0.4 + 5 | 253 | 255 | 261 | 278 | 281 |
| | PVP K30 | 0.4 + 1 | | | | | |
| Dispersion | celecoxib | 20 | 123/ | 127/ | 125/ | 132/ | 130/ |
| 12 | SLS | 0.4 + 5 | 250 | 259 | 255 | 269 | 271 |
| | PVP K30 | 0.4 + 1 | | | | | |
| | sucrose | 20% | | | | | |

The drug loading in the dispersion is an important indicator of the efficiency and effectiveness of manufacturing nanoparticle-based celecoxib formulations. Dispersion 9 and Dispersion 10 in Table 1-2 represent further evaluation of Dispersions 6-7, i.e., the impact of raising celecoxib loading in the dispersion to the drug particle size. This also evaluates how to improve the milling efficiency. Table 1-2 lists partial results in which the celecoxib loading in dispersion is raised from 10% w/w to 20% w/w: the celecoxib particle size in Dispersion 9 is significantly increased. This suggests that the combined use of the surfactant SLS (“sodium dodecyl sulfate”) and the hydrophilic polymer HPMC (“hydroxypropyl methyl cellulose”) is only suitable for milling with low loading, but not for milling with a reasonably high loading. In the meantime, the increase in the amount of SLS and HPMC due to the need for stabilizing celecoxib nanoparticles, also leads to a decrease in flowability of the dispersion (due to increased viscosity), increased foaming, as well as significant increase for temperature inside milling chamber (50-65° C.). The composition of Dispersion 9 was thus abandoned.

Dispersion 10 (see Table 1-2): the particle size of celecoxib remained stable after loading is increased, indicating that SLS and PVP (“polyvinylpyrrolidone”) were of a good combination, and can be further optimized for large-scale milling production. The follow-up study of Dispersion 10 found that although SLS and PVP K30 were a good combination, the particle size was still not sufficiently stable. On Day 3 at ambient temperature, the particle size increased significantly. In large-scale manufacturing, this bolds uncertainty, as there is often a time lagging between the wet-milling and subsequent fluidized-bed drying. The manufacturing process requires that the nanoparticulate dispersion be able to maintain sufficient period of stability.

In Table 1-2, Dispersion 11 utilizes a two-step procedure, i.e., “mill, then stabilize”. That is, both surfactant(s) and hydrophilic polymer(s) are added in two separate portions into the dispersion: one is for wet-milling (Step A), and the other is for stabilizing (Step B). For surfactant (SLS), it is added up to 5.4% w/w in total; for hydrophilic polymer (PVP), it is added up to 1.4% w/w in total. Step B is carried out by mechanical stirring (not wet-milling). From follow-up stability evaluation of the dispersion, celecoxib particle size remains highly stable. It is worth noting that, if no such procedure is practiced (i.e., mill, then stabilize), and if higher concentrations of surfactant (Dispersion 11: SLS is 5.4% w/w) and hydrophilic polymer (polyvinylpyrrolidone: 1.4% w/w) were used all in wet-milling, it would quickly lead to an increase in the viscosity of the dispersion. The excessive surfactant produced much foaming, and the temperature of the milling chamber increased to 50-65° C. (data not listed), all of which would seriously affect the normal operation of the wet-milling process.

Dispersion 12 in Table 1-2 was similar to Dispersion 11. A two-step procedure (“mill, then stabilize”) was used, i.e., under mechanical stirring (non-wet grinding), 5% w/w of SLS, 1% w/w of PVP K30, and 20% w/w of dd sucrose were added into the collected nanoparticulate dispersion 10 (i.e., “Step B”). This made the final concentrations of SLS, PVP K30 and sucrose in nanoparticulate dispersion as: 5.4% and 1.4% and 20% w/w, respectively. The data show that celecoxib particle size in Dispersion 12 upon addition of sucrose is comparable to that in Dispersion 11. The importance of adding sucrose in dispersion is that when preparing solid nanoparticle-based formulations, after the water in the dispersion was removed, sucrose can help celecoxib particles solidify onto the excipients and maintain nanoparticles at stable nanometer range (see the dissolution stability study of celecoxib formulations: Table 2-1, Table 3-1, Table 3-2).

### Example 2

### Preparation of Celecoxib Formulations

I. Composition and manufacturing process for preparing celecoxib formulation (as capsule)

Table 2-1 shows the compositions and manufacturing process of some representative celecoxib formulations (as capsules). A strength of 40 mg celecoxib formulation (loading 160 mg per capsule; capsule size: No. 3) was prepared. Composition 1 and Composition 2 were prepared by fluidized-bed drying via top-spraying, and drug-loaded particles were formed. The fluidized-bed drying equipment and technical parameters were as follows: Diosna Minilab-XP (Germany) for fluidized-bed drying and granulation; inlet air temperature: 65±5° C.; exhaust temperature: 45±5° C.; air volume: 25±5 m³/h; spray rate: 10±5 ml/min. Composition 3 and Composition 4 were prepared by spraying at the bottom of a fluidized-bed, and the drug-loaded spheres were formed. The fluidized-bed drying and granulation process was prepared by FLZB-0.5 fluidized-bed produced by Chuangzhi

Electromechanical Tech Company (Changzhou, China); key technical parameters are: inlet temperature: 60±5° C.; exhaust temperature: 40±5° C.; air volume: 15±5 m³/h; spray rate: 7±2 ml/min.

The manufacturing process for celecoxib formulations (as capsules) is shown in FIG. 3.

TABLE 2-1
Composition and Process for Preparing Celecoxib
Formulations (strength: 40 mg/capsule)

| | Com- | mg/ | | Com- | mg/ | |
|---|---|---|---|---|---|---|
| Steps | ponent | capsule | % | ponent | capsule | % |
| | Composition 1 | | | Composition 2 | | |
| Step A: | cele- | 40 | 25 | cele- | 40 | 25 |
| Preparing | coxib | | | coxib | | |
| nano- | SLS | 0.8 | 0.5 | SLS | 0.8 | 0.5 |
| particulate | PVP | 0.8 | 0.5 | PVP | 0.8 | 0.5 |
| dispersion | K30 | | | K30 | | |
| (wet- | | | | | | |
| milling) | | | | | | |
| Step B: | sucrose | 40 | 25 | | | |
| Stabilizing | SLS | 10 | 6.25 | SLS | 10 | 6.25 |
| the nano- | PVP | 2 | 1.25 | PVP | 2 | 1.25 |
| particulate | K30 | | | K30 | | |
| dispersion | | | | | | |
| (mecha- | | | | | | |
| nical | | | | | | |
| stirring) | | | | | | |
| Step C: | sucrose | 60 | 37.5 | sucrose | 100 | 62.5 |
| Preparing | CCS | 5.6 | 3.5 | CCS | 5.6 | 3.5 |
| celecoxib- | Silica | 0.8 | 0.5 | Silica | 0.8 | 0.5 |
| loaded | | | | | | |
| particles | | | | | | |
| (fluidized- | | | | | | |
| bed | | | | | | |
| drying) | | | | | | |
| | Total | 160 | 100.0 | Total | 160 | 100.0 |
| | weight | | | weight | | |
| | Composition 3 | | | Composition 4 | | |
| Step A: | cele- | 40 | 25 | cele- | 40 | 25 |
| Preparing | coxib | | | coxib | | |
| nano- | SLS | 0.8 | 0.5 | SLS | 0.8 | 0.5 |
| particulate | PVP | 0.8 | 0.5 | PVP | 0.8 | 0.5 |
| dispersion | K30 | | | K30 | | |
| (wet- | | | | | | |
| milling) | | | | | | |
| Step B: | sucrose | 30 | 18.75 | | | |
| Stabilizing | SLS | 10 | 6.25 | SLS | 10 | 6.25 |
| nano- | PVP | 2 | 1.25 | PVP | 2 | 1.25 |
| particulate | K30 | | | K30 | | |
| dispersion | | | | | | |
| (mecha- | | | | | | |
| nical | | | | | | |
| stirring) | | | | | | |
| Step C: | sucrose | 76.4 | 47.75 | Sucrose | 106.4 | 66.5 |
| Preparing | spheres | | | spheres | | |
| celecoxib- | (200- | | | (200- | | |
| loaded | 300 | | | 300 | | |
| spheres | μm) | | | μm) | | |
| (fluidized- | | | | | | |
| bed | | | | | | |
| drying) | | | | | | |
| | total | 160 | 100.00 | total | 160 | 100.0 |
| | weight | | | weight | | |

*: SLS: sodium dodecyl sulfate;
HPMC: hydroxypropyl methylcellulose;
PVP: polyvinylpyrrolidone;
CCS: croscarmellose sodium.
II. Composition and manufacturing process for preparing celecoxib formulation (suspension) The celecoxib formulation (as suspension) was prepared by the following steps (see FIG. 3).
Step A: prepare nanoparticulate dispersion by wet-milling;
Step B: add excipients such as saccharides, as well as a certain portions of surfactant and hydrophilic polymer in order to prepare a stabilized nanoparticulate dispersion. Preferably, mixing is carried out by mechanical stirring.
Step E: add a certain amount of one or more of the following excipients, including: suspending agent, antioxidant, taste masking agent, sweetener, preservative, defoaming agent, thickener, pH buffering salt in solution. The mixing is preferably carried out by mechanical stirring. The manufacturing process of celecoxib formulations (as suspension) is shown in FIG. 3.

Table 2-2 lists representative compositions of celecoxib formulation (as suspension); its strength: 16 mg/ml, 60 ml/bottle.

TABLE 2-2
Composition of Celecoxib Formulation
(as suspension; strength: 16 mg/ml, 60 ml/bottle)

| | Function | mg/ml | % (g/100 ml) |
|---|---|---|---|
| celecoxib | active | 16 | 1.6 |
| SLS | surfactant | 4.32 | 0.432 |
| PVP K30 | hydrophilic | 1.12 | 0.112 |
| | polymer | | |
| sucrose | stabilizer | 50 | 5 |
| anethole | fragrant | 0.25 | 0.025 |
| sodium methyl | preservative | 1.2 | 0.12 |
| paraben | | | |
| sodium propyl | preservative | 0.3 | 0.03 |
| paraben | | | |
| neohesperidin | sweetener | 1 | 0.1 |
| dihydrochalcone | | | |
| pure water | | to the mark | |

### Example 3

### Dissolution Testing of Celecoxib Formulations (as capsule)

Tables 3-1 and Table 3-2 present dissolution data for representative celecoxib formulations (as capsules; see Table 2-1 for detailed composition and process) at 0,1,2,3 months under accelerated stability condition (40° C./75% RH). The dissolution testing was conducted under non-sink conditions, which means that the drug was not fully dissolved the dissolution. This is used mainly to evaluate the impact of different nanoparticle-based formulations and preparation processes on stability (mainly dissolution stability). For example, both Compositions 1 and 3 used sucrose as an important excipient for stabilizing nanoparticulate dispersions in “Step B”, while Compositions 2 and 4 did not use this excipient. The dissolution stability in large part reflects the particle size stability, as whether the nanoparticles are stable, or there forms particle aggregation which causes the decreasing of the drug dissolution. Hence, evaluation of dissolution stability is critical for the absorption of nanoparticle-based formulations in vivo. Tables 3-1 and 3-2 show that Composition 1 was significantly better than Composition 2 in dissolution stability; Composition 3 was better than Composition 4 in that regard.

TABLE 3-1
Dissolution of Celecoxib Formulations (Compositions
1-4; 40 mg/capsule) and Celebrex ® 50 mg/capsules
(stability condition: 40° C./75% RH; 0, 1, 2, 3 months;
dissolution: USP Method 1, basket, 50 rpm, 900 ml,
pH 1.0 diluted hydrochloric acid solution, 37° C.)

| | 40° C./ | | | | | |
|---|---|---|---|---|---|---|
| | 75% | Dissolution of celecoxib (%) | | | | |
| Celecoxib | (month) | 5 | 10 | 20 | 30 | 60 |
| formulations | RH | min | min | min | min | min |
| Composition | 0 | 10.9 | 23.9 | 48.4 | 56.7 | 66.2 |
| 1 | 1 | 11.4 | 25.2 | 47.5 | 53.4 | 62.8. |
| | 2 | 12.1 | 23.3 | 42.2 | 55.1 | 64.3 |
| | 3 | 8.9 | 23.2 | 43.9 | 52.3 | 61.0 |
| Composition | 0 | 6.2 | 21.3 | 38.5 | 47.2 | 52.3 |
| 2 | 1 | 7.2 | 20.2 | 32.5 | 38.4 | 40.2 |
| | 2 | 6.1 | 15.4 | 27.2 | 35.2 | 39.1 |
| | 3 | 5.8 | 14.5 | 25.4 | 27.5 | 35.4 |
| Composition | 0 | 11.2 | 25.5 | 44.2 | 55.2 | 67.3 |
| 3 | 1 | 9.5 | 24.5 | 45.9 | 57.3 | 68.2 |
| | 2 | 11.1 | 23.6 | 42.7 | 54.3 | 62.2 |
| | 3 | 10.1 | 25.7 | 41.2 | 54.1 | 59.1 |
| Composition | 0 | 7.7 | 22.2 | 37.4 | 46.2 | 49.6 |
| 4 | 1 | 8.3 | 20.3 | 34.3 | 40.8 | 45.3 |
| | 2 | 6.3 | 18.2 | 27.9 | 36.1 | 37.9 |
| | 3 | 4.9 | 14.7 | 22.1 | 33.2 | 36.2 |
| Celebrex ® | 0 | 1.7 | 2.7 | 2.9 | 2.9 | 3.1 |
| strength: | 1 | 1.4 | 2.3 | 2.5 | 2.6 | 2.5 |
| 50 mg capsule | 2 | 1.4 | 2.4 | 2.5 | 2.6 | 2.7 |
| Pfizer | 3 | 1.6 | 2.5 | 2.6 | 2.7 | 2.8 |
| Batch: J78193 | | | | | | |

TABLE 3-2
Dissolution of Celecoxib Formulations (Compositions
1-4; 40 mg/capsule) and Celebrex ® 50 mg/capsule
(stability condition: 40° C./75% RH; 0, 1, 2, 3 months;
dissolution: USP Method 1; basket, 50 rpm,
900 ml, pH 6.1 phosphate buffer solution, 37° C.)

| | 40° C./ | | | | | |
|---|---|---|---|---|---|---|
| | 75% | Dissolution of celecoxib (%) | | | | |
| Celecoxib | RH | 5 | 10 | 20 | 30 | 60 |
| formulations | (month) | min | min | min | min | min |
| Composition | 0 | 8.9 | 25.4 | 58.5 | 64.3 | 68.4 |
| 1 | 1 | 6.2 | 24.3 | 60.5 | 62.4 | 66.2 |
| | 2 | 6.8 | 25.3 | 56.9 | 64.4 | 64.6 |
| | 3 | 7.9 | 23.9 | 55.2 | 59.1 | 63.3 |
| Composition | 0 | 6.5 | 19.1 | 37.4 | 45.2 | 54.6 |
| 2 | 1 | 4.5 | 15.4 | 35.7 | 40.3 | 50.2 |
| | 2 | 5.4 | 14.5 | 30.2 | 36.5 | 48.3 |
| | 3 | 4.7 | 15.3 | 20.5 | 32.4 | 39.5 |
| Composition | 0 | 10.2 | 27.4 | 63.2 | 69.3 | 69.4 |
| 3 | 1 | 7.9 | 25.7 | 62.4 | 65.4 | 68.3 |
| | 2 | 8.2 | 22.7 | 59.3 | 67.3 | 64.6 |
| | 3 | 7.2 | 23.5 | 56.3 | 62.2 | 62.9 |
| Composition | 0 | 5.5 | 20.4 | 40.5 | 47.2 | 57.2 |
| 4 | 1 | 6.3 | 18.4 | 37.2 | 44.3 | 52.4 |
| | 2 | 4.7 | 14.2 | 33.2 | 39.5 | 44.5 |
| | 3 | 6.5 | 11.2 | 22.8 | 33.1 | 43.2 |
| Celebrex ® | 0 | 1.4 | 2.4 | 2.9 | 3 | 2.9 |
| Strength: 50 mg | 1 | 1.3 | 2.1 | 2.6 | 2.6 | 2.5 |
| capsule | 2 | 1.2 | 2.2 | 2.5 | 2.6 | 2.8 |
| Pfizer | 3 | 1.1 | 2.1 | 2.4 | 2.5 | 2.4 |
| Batch: J78193 | | | | | | |

### Example 4

A Comparative Pharmacokinetic Study of Celecoxib Formulation with Celebrex® in Beagle Dogs The pharmacokinetic study of celecoxib was conducted in non-naive male and female beagle dogs. The mode of administration: oral, single dosing. There were three groups: Celecoxib Test Formulation 1 (50 mg/capsule); Celecoxib Test formulation 2 (25 mg/capsule); commercial product Celebrex (100 mg/capsule). The pharmacokinetic parameters were analyzed to evaluate in vivo performance of celecoxib formulations and Celebrex. The composition of the celecoxib formulation in this experiment was consistent with Composition 1 in Table 2-1, but the capsule size and filling volume were adjusted accordingly in proportion.

Experimental: the study used two male and two female non-naive beagle dogs, weighing 9-11 kg. Each animal was marked on ear with a unique number tattoo. Four animals were dosed orally in the following order: Celecoxib Test Formulation 1 (50 mg/capsule/beagle), Celebrex (100 mg/capsule/beagle), Celecoxib Test Formulation 2 (25 mg/capsule/beagle). Blood samples were collected from each animal at the same preset time point.

Preparation and dosing: all animals for dosing were pre-weighed and recorded. Sample collection and preparation: Serial blood samples (approximately 0.5 mL in K2EDTA) were collected via a peripheral vessel. Blood samples were collected at pre-dose (0 minute) and post-dose at 10, 20, 30, 45 minutes, 1, 1.25, 1.5, 2, 3, 4, 6, 8, 12, 24 and 48 hours for all beagle dogs of the 3 Phases. After collection, blood samples were gently inverted several times and immediately placed on wet ice prior to centrifugation at 2 to 8° C. and 3000 g for 10 minutes. About 0.2 mL plasma was harvested and transferred into a pre-labeled transparent vial, and stored frozen at −60° C. or lower until shipped on dry ice for analysis.

Clinical observation: twice daily (approximately 9:30 a.m. and 4:00 p.m.), cage-side observations for general health and appearance were performed. Beagle dogs were given physical examination prior to study initiation to confirm their health. On dosing days, the beagle dogs were observed before and after each sample collection time point. General condition, behavior, activity, excretion, respiration or other unusual observations noted throughout the study was recorded in the raw data.

The beagle dog plasma samples were analyzed for celecoxib using a qualified bioanalytical method based on protein precipitation followed by LC/MS/MS analysis. The lower limit of quantification (LLOQ) for celecoxib in plasma was 1.00 ng/mL and the upper limit of quantification (ULOQ) in plasma was 3000 ng/mL.

Pharmacokinetic data analysis: plasma concentration data of celecoxib were subjected to a non-compartmental pharmacokinetic analysis using WinNonlin Version 6.2.1 (Pharsight, Mountain View, Calif.). Peak plasma concentrations (Cₘₐₓ) and the corresponding peak times (Tₘₐₓ) were taken directly from the plasma concentration vs. time profiles.

Terminal half-life (t_{1/2}), mean residence time (MRT) from time zero to infinity (MRT_{0·inf}), mean residence time (MRT) from time zero to the last quantifiable concentration (MRT_{0·last}) the area under the plasma concentration-time curve (AUC) from time zero to the last quantifiable concentration (AUC_{0·last}) and AUC from time zero extrapolated to infinity (AUC_{0·inf}) were calculated using the model of linear log trapezoidal.

All pharmacokinetic parameters such as Cₘₐₓ, Tₘₐₓ, AUC, t_{1/2} and MRT values were reported to three significant figures. For samples collected within the first hour of dosing, all were collected a ±1 minute. For the remaining time points, all were taken within 5% of the scheduled time. So nominal sampling times were used to calculate pharmacokinetic parameters since in no situations were there a deviation in sample collection.

Table 4 shows the mean values of main pharmacokinetic parameters obtained by single oral dosing of celecoxib formulations and the reference formulation Celebrex® to non-naive beagle dogs, including Cₘₐₓ, Tₘₐₓ, AUC₀₋ₗₐₛₜ, AUC_{0-inf}, AUC_{Extrap}(%), t_{1/2}, MRT₀₋ₗₐₛₜ and MRT_{0-inf}.

In this study, two celecoxib formulations were dosed at 25 mg and 50 mg per capsule per dog, to a group of two male beagle dogs and two female beagle dogs. Cₑlₑbᵣₑₓ® (100 mg per capsule), the celecoxib commercial product, was dosed at 100 mg per capsule per dog to the same four beagle dogs. There was at least one week washout period between each phase. The dosing was arranged in the sequence as follows:
Phase 1: Celecoxib Test Formulation 1 (50 mg per capsule per beagle dog)
Phase 2: Celebrex® (100 mg per capsule per beagle dog)
Phase 3: Celecoxib Test Formulation 2 (25 mg per capsule per beagle dog)

TABLE 4
PK Study of Celecoxib Formulations and Celebrex ®
Following Single Oral Dosing to Non-naive Beagle Dogs

| | Phase | | |
|---|---|---|---|
| | 1 | 2 | 3 |
| | Celecoxib Test | Celebrex ® | Celecoxib Test |
| PK | Formulation 1 | (100 mg/ | Formulation 2 |
| Parameters | (50 mg/capsule) | capsule) | (25 mg/capsule) |
| Cₘₐₓ | 1131 ± 254 | 662 ± 363 | 915 ± 89.6 |
| (ng/mL) | | | |
| Tₘₐₓ (h) | 1.75 ± 1.09 | 0.917 ± 0.382 | 0.833 ± 0.577 |
| AUC₀₋ₗₐₛₜ | 7720 ± 1204 | 5293 ± 852 | 5153 ± 2284 |
| (ng/mL · hr) | | | |
| AUC_{0-inf} | 7797 ± 1170 | 5337 ± 840 | 5187 ± 2269 |
| (ng/mL · hr) | | | |
| AUC_{Extrap} | 1.04 ± 0.662 | 0.831 ± 0.473 | 0.771 ± 0.586 |
| (%) | | | |
| t_{1/2} (h) | 5.60 ± 1.49 | 7.26 ± 1.32 | 5.59 ± 1.12 |
| MRT₀₋ₗₐₛₜ | 8.87 ± 2.67 | 11.0 ± 3.97 | 7.13 ± 2.46 |
| (h) | | | |
| MRT_{0-inf} (h) | 9.21 ± 2.61 | 11.3 ± 4.15 | 7.40 ± 2.42 |

FIG. 4 shows the mean plasma concentration of celecoxib from the present disclosure (magnified curve: 0-12 hours), which was obtained by single oral dosing of Celecoxib Test Formulation 1 (50 mg per capsule per beagle dog), Celecoxib Test Formulation 2 (25 mg per capsule per beagle dog), and Celebrex® (100 mg per capsule per beagle dog) to male and female beagle dogs.

Celecoxib Test Formulations in Table 4 show significantly increased exposure (Cₘₐₓ, AUC) as compared to those of the Celebrex: the C. for Celecoxib Test Formulations 25 mg, 50 mg and Celebrex® 100 mg are as follows respectively: 915 ng/mL, 1131 ng/mL, and 662 ng/mL; the AUC_{0-inf} for Celecoxib Test Formulations 25 mg, 50 mg and Celebrex® 100 mg are as follows respectively: 5187 ng/mL·hr, 7797 ng/mL·hr, and 5337 ng/mL·hr.

The data show that the ratios of the geometric mean of C. and AUC for Celecoxib Test Formulation 1 (25 mg per capsule) vs. Celebrex® (100 mg per capsule) were 1.38 and 0.97, respectively, while the dosing amount for Celebrex (100 mg per capsule) was 4-folds as that for Celecoxib Test Formulation 1 (25 mg per capsule).

Also, the AUC_{0·inf} and Cₘₐₓ of the Celecoxib Test Formulation 2 (50 mg per capsule) were increased by 1.50-folds and 1.24-folds, respectively, as compared with those of Celecoxib Test Formulation 1 (25 mg per capsule). The increasing trends remain, though they are not in full proportion with dosing.

### Example 5

A Pharmacokinetic Study of Celecoxib Formulation AP2500 and Celebrex in Healthy Humans

This example describes a comparative pharmacokinetic study of celecoxib formulations AP2500 in 3 doses (50 mg, 75 mg, 100 mg) and the reference Celebrex® (100 mg) in healthy subjects. The study was conducted in a single clinical center, with a design as: fasting, single dose, four-period, four-treatment, and crossover (4x4). The celecoxib formulation used in this study was consistent with Composition 1 in Table 2-1, with the capsule size and filling weight being adjusted accordingly in proportion with the strength. A total of 12 eligible subjects were selected and randomly divided into four groups A, B, C, and D, with 3 cases in each group. In each cycle, the subjects were given light diets the day before the experiment. After an overnight fast (free water) for at least 10 hours, the subjects were given the following drugs along with 240 mL of warm water in accordance with the random schedule and administration method:
Formulation 1 (T₁): Celecoxib formulation AP2500 capsules 50 mg
Formulation 2 (_{T2}): Celecoxib formulation AP2500 capsules 75 mg
Formulation 3 (T₃): Celecoxib formulation AP2500 capsules 100 mg
Reference formulation (R): Celebrex® Capsules 100 mg
The washing period was 1 week.

The administration schedule was shown in Table 6. Water was forbidden from 1 hour before administration to 1 hour after administration, and free water were given at other times. The subjects were fasted within 4 hours after the administration. The subjects in each cycle were given standard diets at the same specific time point (4 hours and 10 hours after the administration).

Other matters that require the subject's cooperation during the research period include: avoiding strenuous activities after the administration, and not staying in bed for a long time. During the research period, the subjects' diet as well as work/rest time were arranged uniformly. Any food and beverages containing alcohol and xanthenes were forbidden, such as chocolate, tea, coffee and cola, etc. Smoking and drinking grapefruit juice or products containing grapefruit were prohibited. The subjects followed the protocol and any drugs was forbidden (including the washing period). The exception was that drugs must be used in the treatment of sudden diseases and should be notified in time.

TABLE 5
Schedule for administration of celecoxib formulation AP2500

| | Treatment A | Treatment B | Treatment C | Treatment D |
|---|---|---|---|---|
| | (T₁T₂T₃R) | (T₂T₃RT₁) | (T₃RT₁T₂) | (RT₁T₂T₃) |
| Cycle | Formulation 1 | Formulation 2 | Formulation 3 | Reference |
| 1 | | | | formulation |
| | | | | (R) |
| Cycle | Formulation 2 | Formulation 3 | Reference | Formulation 1 |
| 2 | | | formulation | |
| | | | (R) | |
| Cycle | Formulation 3 | Reference | Formulation 1 | Formulation 2 |
| 3 | | formulation | | |
| | | (R) | | |
| Cycle | Reference | Formulation 1 | Formulation 2 | Formulation 3 |
| 4 | formulation | | | |
| | (R) | | | |

Blood sample collection and processing: 4 ml of venous blood (in the EDTA anticoagulation tube) was collected for Treatments A, B, C, and D before the administration (0 hours) and 0.5, 1, 1.5, 2, 2.5, 3, 3.5, 4, 5, 6, 8, 12, 24, 36, 48 hours after the administration, totally 16 points. After the blood sample was collected, it was centrifuged at 3000 rpm for 10 minutes, and the plasma was collected and stored below -70° C. for later use.

Sample analysis: the drug concentration of celecoxib in plasma was determined by HPLC-MS/MS method.

Data processing: WINNONLIN program was used to calculate the main pharmacokinetic parameters and perform equivalent analysis. The pharmacokinetic parameters were calculated based on the actual blood collection time.

The results were shown in Table 6.

TABLE 6
Summary of PK Parameters of Celecoxib in Healthy Volunteers after Single Oral
Administration of Treatment A, B, C and D under Fasting Conditions

| | | | | | Reference |
|---|---|---|---|---|---|
| | | Test: AP2500 | | | Celebrex ® |
| | | Treatment A | Treatment C | Treatment D | Treatment B |
| | | T1 (50 mg) | T2 (75 mg) | T3 (100 mg) | R (100 mg) |
| PK Parameter | Statistics | N = 12^{a} | N = 11 | N = 11 | N = 11 |
| Cₘₐₓ(ng/mL) | Mean (SD) | 426 (91.4) | 533 (155) | 784 (243) | 568 (185) |
| Tₘₐₓ (h) | Median | 2.00 | 2.50 | 2.50 | 2.00 |
| | (range) | (1.00-5.00) | (1.50-3.50) | (1.00-4.02) | (1.00-4.00) |
| AUC₀₋ₜ | Mean (SD) | 1750 (393) | 2760 (758) | 3760 (1080) | 3290 (826) |
| (ng · h/mL) | | | | | |
| AUC_{0-∞} | Mean (SD) | 1820 (406) | 2840 (746) | 3840 (1090) | 3410 (792) |
| (ng · h/mL) | | | | | |
| AUC₀₋ₜ/AUC_{0-∞} | Mean (SD) | 0.960 | 0.970 | 0.980 | 0.963 |
| | | (0.0182) | (0.0274) | (0.00960) | (0.0577) |
| λz (1/h) | Mean (SD) | 0.137 | 0.126 | 0.121 | 0.102 |
| | | (0.0640) | (0.0301) | (0.0261) | (0.0401) |
| t_{1/2} (h) | Mean (SD) | 5.75 (1.76) | 5.78 (1.24) | 5.95 (1.17) | 8.16 (4.91) |
| F_{Rel}^{b} | Mean (SD) | 1.08 (0.112) | 1.12 (0.178) | 1.13 (0.208) | NA |

NA: Not applicable
^{a}one subject in Treatment A T1 withdrew from the trial 12 hours after blood collection
${b}^{} {F}_{Re l} = \frac{{AUC}_{0 - ∞ Test}}{{AUC}_{0 - ∞ Reference}} \times \frac{{Dose}_{Reference}}{{Dose}_{Test}}$

The data in Table 6 show that the PK parameters of AP2500 at a dose of 50 mg to 100 mg were largely proportional to the dose, and there was a linear kinetic trend. Compared with the reference (Celebrex® 100 mg), for AP2500 (50 mg) in the T1 group of celecoxib formulation, the area values (AUC₀₋ₜ and AUC_{0-∞}) under the plasma concentration-time curve were 53.52% and 53.59%, and the peak concentration was (77.07%).For AP2500 (75 mg) in the T2 group of celecoxib formulation, the area values (AUC₀₋ₜ and AUC_{0-∞}) under the plasma concentration-time curve were 83.71% and 83.93%, and the peak concentration was 94.58%.For AP2500 (100 mg) in the T3 group of celecoxib formulation, the area values (AUC₀₋ₜ and AUC_{0-∞}) under the plasma concentration-time curve were 113.56% and 111.39%, and the peak concentration was 13 8%.

### Equivalence

The present disclosure discloses a new celecoxib formulation and preparation method thereof, for treating mild to moderate acute pain and chronic pain. Some specific embodiments are listed, which are intended to illustrate rather than limit the claims. After reading, those skilled in the art can easily implement and make some changes. The full scope covered by the present disclosure is defined by the claims.

### Reference Description

Although a number of examples of the present disclosure have been described, it should be understood that these examples are only illustrative and not restrictive, and many variations are obvious to those skilled in the art. In addition, the steps can be performed in any suitable order (and any foreseeable steps can be added and/or any foreseeable steps can be eliminated).

We claim:

1. A celecoxib oral formulation, wherein its strength is at 60-90% of the original strength of the commercial celecoxib product, and the formulation is bioequivalent to the commercial celecoxib product.

2. The celecoxib formulation according to claim 1, its dosage form includes tablets, capsules, granules, or suspension.

3. The celecoxib formulation according to claim 1, the particle size of the celecoxib has a D50 of not greater than 160 nm and a D90 of not greater than 300 nm.

4. The celecoxib formulation according to claim 1, wherein the formulation is in the dosage form of tablets, capsules or granules, and wherein the formulation comprises sodium dodecyl sulfate at 0.5-12% w/w, preferably 2-10% w/w, more preferably 4-8% w/w, based on total weight calculation of the formulation.

5. The celecoxib formulation according to claim 1, wherein the formulation is in the form of tablets, capsules or granules, and wherein the formulation comprises polyvinylpyrrolidone at 0.5-7% w/w, preferably 0.5-5% w/w, more preferably 0.5-3% w/w, based on total weight calculation of the formulation.

6. The celecoxib formulation according to claim 5, wherein the dosage is in the form of tablets, capsules or granules, and wherein the formulation comprises sucrose at 10-70% w/w, preferably 10-50% w/w, more preferably 10-30% w/w, based on total weight calculation of the formulation.

7. The celecoxib formulation according to claim 1, wherein the dosage is in the form of tablets, capsules or granules, and the strength includes: 40 mg, 80 mg, 160 mg, 320 mg; which respectively correspond to the strength of the commercial celecoxib product: 50 mg, 100 mg, 200 mg, 400 mg.

8. The celecoxib formulation according to claim 1, wherein the dosage is in the form of tablets, capsules or granules, and the formulation further comprises one or more pharmaceutical acceptable excipients selected from the group consisting of fillers, disintegrants, binders, glidants, and lubricants.

9. The celecoxib formulation according to claim 1, wherein the dosage is in the form of tablets, capsules or granules, and with a strength in the range of 40-80 mg, the dissolution of celecoxib is not less than 30% in 30 minutes and not less than 45% in 60 minutes, using the USP Dissolution Method I, measured in a dissolution medium at pH 1.0 or at pH 6.1, both of which are at 50 rpm.

10. The celecoxib formulation according to claim 1, wherein the dosage in the form of suspension, and the formulation comprises celecoxib at 0.5-5% w/v, preferably 0.5-3% w/v, more preferably 1-2% w/v, based on total weight calculation of the formulation.

11. The celecoxib formulation according to claim 1, wherein the dosage is in the form of suspension, and wherein the formulation comprises sodium dodecyl sulfate at 0.1-2% w/v, preferably 0.1-1.5% w/v, more preferably 0.1-1% w/v, based on total weight calculation of the formulation.

12. The celecoxib formulation according to claim 1, wherein the dosage in the form of suspension, and wherein the formulation comprises polyvinylpyrrolidone at 0.05-2% w/v, preferably 0.05-1% w/v, more preferably 0.05-0.5% w/v, based on total weight calculation of the formulation.

13. The celecoxib formulation according to claim 1, wherein the dosage is in the form of suspension, and wherein the formulation comprises sucrose at 0.5-30% w/v, preferably 0.5-20% w/v, more preferably 0.5-10% w/v, based on total weight calculation of the formulation.

14. A method for preparing the celecoxib oral formulation according to any one of claims 1-9, wherein the dosage is in the form of tablets, capsules or granules, and the method comprises the following steps:
Step A: prepare celecoxib into a nanoparticulate dispersion by an all-aqueous wet-milling process; wherein sodium dodecyl sulfate is used as a surfactant, and polyvinylpyrrolidone is used as a hydrophilic polymer;
Step B: add saccharides to the nanoparticulate dispersion obtained from Step A; add more sodium dodecyl sulfate and more polyvinylpyrrolidone; mix to obtain a stabilized nanoparticulate dispersion; one or two or more saccharides are selected from a group consisting of monosaccharides, disaccharides, and polyols; preferably one or two or more saccharides are selected from a group consisting of lactose, sucrose, fructose, mannitol, and sorbitol; and wherein preferably mixing is carried out by mechanical stirring;
Step C: prepare drug-loaded particles or drug-loaded spheres or drug-loaded powder using a fluidized-bed drying process by spraying the nanoparticulate dispersion obtained from Step B onto the carriers;
Step D: prepare an oral solid dosage form such as tablets, capsules, or granules, using the above drug-loaded particles or drug-loaded spheres or drug-loaded powder.

15. The preparation method according to claim 14, in the wet-milling process in Step A, the nanoparticulate dispersion comprises celecoxib at more than 10% w/w, preferably 10-35% w/w, more preferably 15-25% w/w, based on weight calculation.

16. The preparation method according to claim 14, upon completing Step B, based on weight calculation, the nanoparticulate dispersion comprises sodium dodecyl sulfate at 0.5-12% w/w, preferably 2-10% w/w, and more preferably 4-8% w/w; the nanoparticulate dispersion comprises polyvinylpyrrolidone at 0.5-7% w/w, preferably 0.5-5% w/w, and more preferably 0.5-3% w/w; the nanoparticulate dispersion comprises sugar at 10-70% w/w, preferably 10-50% w/w, and more preferably 10-30% w/w.

17. The preparation method according to claim 14, wherein Step C uses carriers as for filler, which includes saccharides, or spheres of 100-1000 μm; wherein one or two or more saccharides used are selected from a group consisting of monosaccharides, disaccharides, and polyols, and wherein spheres used are selected from a group consisting of sucrose spheres, microcrystalline cellulose spheres, starch spheres, lactose spheres, silicon dioxide spheres, hypromellose spheres, citric acid spheres, or tartaric acid spheres.

18. The preparation method according to claim 17, wherein the carrier further comprises one or two or more excipients selected from disintegrant, binder, glidant, lubricant, and antioxidant.

19. A preparation method for the celecoxib oral formulation according to any one of claims 1-3, 10-13, wherein the formulation is in a dosage form of suspension, and the method comprises Step A, Step B and Step E:
Step A: prepare celecoxib into a nanoparticulate dispersion by an all-aqueous wet-milling process; wherein sodium dodecyl sulfate is used as a surfactant, and polyvinylpyrrolidone is used as a hydrophilic polymer;
Step B: add saccharides to the nanoparticulate dispersion obtained from Step A, continue to add more sodium dodecyl sulfate and more polyvinylpyrrolidone; mix to obtain a homogeneous nanoparticulate dispersion; wherein one or two or more saccharides used are and selected from a group consisting of monosaccharides, disaccharides, and polyols; preferably one or two or more selected from the group consisting of lactose, sucrose, fructose, mannitol, and sorbitol; and wherein preferably mixing is carried out by mechanical stirring;
Step E: add one or two or more of the following pharmaceutical excipients to the nanoparticulate dispersion obtained from Step B, including suspending agent, antioxidant, taste masking agent, sweetener, preservative, defoamer, thickener, fragrant, pH buffering salt; preferably mixing is carried out by stirring.

20. Use of the celecoxib oral formulation according to any one of the claims 1-13, for treating mild to moderate acute pains, mild to moderate chronic pains; or for the preparation method for celecoxib formulation for treating mild to moderate acute pains, and mild to moderate chronic pains.
